(19) United States
(12) Patent Application Publication
(10) Pub. No.: US 2007/0243304 A1
(43) Pub. Date: Oct. 18, 2007

(54) Omega-3 Fatty Acid Compositions With Honey

(76) Inventors: Mahabaleshwar Hedge, Pune (IN); Ulhas Wagh, Pune (IN); Shahida Khan, Pune (IN)

Correspondence Address: LADAS & PARRY, 26 WEST 61ST STREET, NEW YORK, NY, 10023, US

(21) Appl. No.: 11/597,302

(22) PCT Filed: May 24, 2005
(86) PCT No.: PCT/IN05/00163

§ 371 Date: Nov. 22, 2006

(30) Foreign Application Priority Data
May 24, 2004 (IN) 587/MUM/2004

(51) Int. Cl. A61K 31/19; A61K 35/64

(52) U.S. Cl. 426/593; 426/533; 426/541; 426/72

(57) ABSTRACT

A synergistic composition consisting of Omega-3 fatty acids, Honey and Natural antioxidants, and a process to prepare such a composition is disclosed. The composition is flavored if desired. The synergistic preparation abounds in antioxidants to prevent the omega-3 fatty acid from being oxidized and to provide additional antioxidants to the body. The composition is biocompatible and stable for six months. The composition can be used as a vehicle to incorporate other essential nutrients and herbal extracts having medicinal properties.

### FIELD OF THE INVENTION

The present invention relates to synergistic composition(s) containing Omega-3 fatty acids and antioxidants in organic honey. These compositions can be further utilized for obtaining nutraceuticals and herbal pharmaceutical preparations including beverages.

### BACKGROUND ART

Omega-3 fatty acids are essential fatty acids, because they can't be synthesized by the human body and are necessary for normal metabolic activities leading to optimum physical and mental health. Moreover the modern diet is very deficient in these vital nutrients. Therefore it is absolutely essential to provide these nutrients in the daily diet. Alpha-linolenic (ALA) acid is the parent omega-3 essential fatty acid while linoleic acid (LA) is the parent omega-6 essential fatty acid. Intake of omega-3 and omega-6 fatty acids in the diet should be in a balanced ratio for good health. The modern diet has too much omega-6, in the form of LA, disrupting the essential fatty acid balance and promoting disease. Supplementing the diet with omega-3 fatty acids can restore proper balance and ensure good health

The need for Omega-3 fatty acids has been extensively documented.

A global dietary shift, unprecedented in human history favoring the ingestion of excessive omega-6 fatty acids at the expense of omega-3 fatty acids, with concomitant decrease in antioxidant intake, is owed as a primary, if not the leading cause, of several degenerative diseases. Omega-3 Fatty acids and anti-oxidants are now regarded as endangered nutrients. There is a critical need for omega-3 fatty acid, which must be met by supplementation (Jade Beutler, “Life in Balance—The critical need for omega-3 supplementation” Natural Medicine, Online July, 2000)

Modern methods of animal husbandry were developed to purposely fatten livestock for slaughter, who have been fed with grains rich in omega-6 and devoid of omega-3. Eggs, chicken, cattle, milk, and even fish have become deficient in omega-3 fatty acids.

Refined food manufacturers were quick to learn that omega-3 fatty acids significantly decreased the shelf life and therefore the marketability of their products. All this has lead to further decrease in the levels of omega-3 in the modern diet. With simultaneous rise in omega-6 fat, the imbalance is more pronounced with dire consequence to human health.

The present increase in severity and rise in incidence of stress related degenerative diseases is largely attributed to the depletion of omega-3 fatty acids and antioxidants in the modern diet. These are now regarded as endangered nutrients. Decosahexanoic acid (DHA) an omega-3 fatty acid is an important constituent of human brain and retina. Eicosapentanoic acid (EPA) is another omega-3 fatty acid from which many anti inflammatory eicosonids—prostaglandins, throboxanes, leukotriens are derived. These carry out hundreds of useful functions in the body. Alpha linolenic acid (ALA) is the parent omega-3 fatty acid that is available from plant sources. ALA (18:3) can be elongated and from these highly unsaturated EPA (20:5) and DHA (22:6), can be derived in the human body. (Artemis Simopoulos, “Essential fatty acids in health and chronic disease” Am J Clin Nutrtion, 560S, 1999.)

Modern man is subjected to higher levels of oxidative stress and his diet is depleted of antioxidants. Poly unsaturated fatty acids being very labile are prone to oxidation. To make the omega-3 fatty acids stable and also make them more effective in the human body, it is thought to be very appropriate to give these two vital nutrients together to give synergistic effect. (Artemis Simopoulos in “Antioxidant status, diet, nutrition and health” CRC Press New York, 1999.)

Omega-3 fatty acid supplementation has been shown to improve many disease conditions including cardiovascular diseases, inflammatory, immunological diseases and cancer. (Gary Wikkholm, “Essential fatty acids: Review of clinical Applications”, Natural Medicine Online June 2000.)

Omega-3 fatty acids are important components of infant formulae and as such are important both for maternal and infant nutrition. They have been shown to be useful in the treatment of mental disorders including Schizophrenia, and Attention Deficit Hyperactivity Disorder (ADHD) (Jade Beutler, “Life in Balance—The Critical need for omega-3 supplementation” Natural Medicine Online July 2000.)

The patents in the field are mainly concerned with uses of Omega-3 fatty acids and making formulations of Omega-3 fatty acids as food supplements for specific disease conditions or actual methods of formulation for increasing the stability and shelf life or bioavailability.

U.S. Pat. No. 4,678,808 describes the use of an emulsion of marine fluids to administrating intravenously to treat thrombotic disease states.

U.S. Pat. No. 5,225,441 describes compositions for treating gingivitis and periodontitis. Omega-3 polyunsaturated fatty acids compete with omega-6 polyunsaturated fatty acids as a substrate in the arachidonic acid cascade and can therefore alter the synthesis of prostaglandin and leukotrienes, both of which are powerful mediators of inflammation and immune response.

Other uses of omega-3 fatty acid oils are disclosed in prior art such as in U.S. Pat. No. 5,034,415 (diabetes mellitus), U.S. Pat. No. 4,879,312 (enhancing angiogenesis), JP 1290625 (improvement of cerebral function), EP 378, 824 (anti-cachexia, cholesterol and triglyceride levels reduction, platelet aggregation inhibition, colon adenocarcinomas growth inhibition), U.S. Pat. No. 5,457,130 (cancer cachexia, malignant tumors, abnormal cAMP levels in adipose tissue, lipolytic activity inhibition), U.S. Pat. No. 5,436,269 (hepatitis) U.S. Pat. No. 6,136,795 (Arthritis), U.S. Pat. No. 5,968,896 (Pre operative feeding), and U.S. Pat. No. 5,766,621 (support of pediatric patients).

U.S. Pat. No. 6,723,357 by Shifferaw et al discloses a high-energy food product. The patent uses honey as a binder or a medium to keep the ingredients together. U.S. Pat. No. 6,284,268 by Mishra et al. discloses pharmaceutical compositions, which contain Omega-3 fatty acid oil. The patent is concerned with preparation of microemulsion of cyclosporin with omega-3 fatty acid oil using a surfactant system.

U.S. Pat. No. 6,274,751 by Tacconi discloses procedures for preparation and use of wax esters enriched in omega-3 unsaturated fatty acids.

U.S. Pat. No. 6,579,544 by Rosenberg et al. discloses a method for supplementing a diet using a blend containing omega-3 fatty acid from flax oil with cartilage protectors such as glucosamine or chondroitin, vitamins, minerals etc.

U.S. Pat. No. 6,344,482 by Stoll et al discloses the use of omega-3 fatty acids in the treatment of bipolar disorder.

U.S. Pat. No. 4,963,380 by Schroeder et al discloses preparation of beverages containing fish oils using fructose as a stabilizer. The fructose is from the high fructose corn syrup or maple syrup. Moreover the preparation uses BHT, BHA and BHQ, the synthetic oil soluble antioxidants, whereas the present invention avoids these synthetic antioxidants and prefers to use the naturally found antioxidants vitamin C and vitamin E and inorganic antioxidants like sodium metabisulphite, and the phenols and the antioxidant naturally occurring in honey for stabilizing and better utilization of the omega-3 fatty acids in a human body.

Russian Patent No. RU 2163079 discloses some versions of preparation of food supplements where flax is used in one of the versions and honey is used in another version. The patent is more concerned with production of a mix of nutrients.

In response to the decreasing availability of concentrated food sources containing essential fatty acids and vitamin E and other antioxidants, health professionals have recommended the development and production of low fat foods rich in these important nutrients.

One method of modifying food products is fortification in a particular medium. In fact, the ability to modify the amount of essential fatty acids and antioxidants through dietary manipulation provides an exciting opportunity to improve the quality of food supply.

However this poses problems, as omega-3 fatty acids are unstable in nature, as they are prone to oxidation. For example, in case of Omega-3 fatty acids from flax oil, the level of peroxides deteriorates very fast: with the formation of lipid peroxides, if left unused. This is shown in table 1 below.

TABLE 1
Lipid Peroxidation of Flax Oil (n moles of malanodialdehydes (MDA))

| Day | Oil |
|---|---|
| 0 | 0 |
| 30 | 1.096 |
| 60 | 5.67 |
| 90 | 8.36 |
| 120 | 13.8 |
| 150 | 28.1 |
| 180 | 36.03 |
| 210 | 47.73 |

Oxidation and rancidity resulting therefrom must be avoided.

The present invention provides a means of taking natural omega-3 fatty acids and antioxidants in a stable form, in one's daily routine, for the health benefits.

An aspect of the invention is to provide both, antioxidants and Omega-3 fatty acids together in a nutritional composition in a natural and stable form.

Another aspect of this invention is to have a composition, which protects the nutraceutically important highly labile omega-3 fatty acid molecules from oxidation and also from the damaging effect of light and air.

Yet another aspect of this invention is to have a composition of Omega-3 fatty acid molecules, which may be easily dispensed and packaged so as to protect the omega-3 fatty acid molecules from deterioration during processing, transport and actual use.

### SUMMARY OF INVENTION

The present invention relates to nutraceutical compositions containing healthy essential omega-3 fatty acids sourced from flax, though the invention is not limited to Omega-3 derived from flax. The source for the Omega-3 fatty acid component in compositions disclosed in the specification may be obtained from other plants, algae, fungi, fish or egg or combinations thereof. The organic honey and natural antioxidants for the compositions are obtained from different sources. These compositions can be further utilized for obtaining nutraceutical and herbal pharmaceutical preparations including beverages.

The various natural antioxidants in the composition protect, the highly labile omega-3 fatty acid molecules from oxidation. The composition(s) are dispensed and packaged to protect the composition(s) from deterioration resulting from, inter alia, light and air during processing, transport and actual use. The omega-3 fatty acid in this formulation is very palatable. It can be used as such. It can also be further modified to include different flavors suiting different tastes. It can also be mixed with different beverages such as milk, yogurt, fruit juice, tea etc, for daily consumption as a natural routine drink, to meet the daily needs of omega-3 fatty acids and antioxidants.

### BRIEF DESCRIPTION OF THE FIGURE

The FIGURE shows the stability of an omega-3 compositions of the invention.

### DETAILED DESCRIPTION OF THE INVENTION

For preparing various compositions, a plethora of natural omega-3 fatty acid oil sources are available such as common oil seeds like flax and chia, marine algae, some fungi, fish oil, egg yolk etc., which are rich in omega-3 fatty acids such as alpha linolenic acid (ALA), ecosapentanoic acid (EPA), and Decosa hexanoic acid (DHA). However, by nature omega-3 fatty acid oils are very labile, because of which, the shelf life of these oils is significantly decreased.

The fish oils are the by-products of the fishing industry and are a direct source of EPA and DHA. Marine algae and certain fungi are also good sources of omega-3 fatty acid oils. However, omega-3 oil is destroyed by peroxidation when exposed to heat, light and air. Fish oils must undergo complex refining to reduce odor, peroxides, free fatty acids and potential toxins due to contaminants such as DDT and mercury. Moreover, the cost of omega-3 from fish source can be about 3-4 times more than that from vegetable sources. It has also been reported that fish may not be a sustainable resource of omega-3 fatty acids, whereas omega-3 fatty acids from vegetable sources including marine algae, would be sustainable.

For describing a preferred embodiment, the present invention uses flax as a source for Omega-3 fatty acids. It is, however, for the purpose of illustration and not limitation of the invention. The method described here can be used for the stabilization of omega-3 fatty acids from other sources.

A process for preparing a composition of omega-3 fatty acid with Honey and natural antioxidants comprises:
extraction of omega-3 fatty acids;
emulsifying omega-3 fatty acids with honey using suitable surfactants;
enriching the omega-3 fatty acids, honey mixture with natural antioxidants; and
flavouring the composition with flavours or fruit powders, to form a synergistic and stable composition.

The omega-3 fatty acids may be extracted from Flax seeds but the invention is not limited to extraction of omega-3 fatty acids from flax seeds. The omega-3 fatty acids may be extracted from plant, algae, fish or eggs or any biological source or combination thereof.

The honey which is unifloral or multifloral, has a moisture content between 17-27% and reducing sugar in the honey is between 67-77%, whereas the fructose to glucose ratio is between 0.95-1.35. Suitable surfactants for use in the present invention are selected from Polysorbate (40, 60, 80), Pluronic acid etc. In the present invention, the honey contains natural antioxidants and those are Vitamin E, Vitamin C and the said antioxidant is at least one. The composition may contain at least water or lipid soluble antioxidants. The composition may also include photochemicals or flavonoids. The compositions are stable for longer than six months when kept at temperatures of from 4-10° C. The Synergistic composition is kept in a stabilized and emulsified state for at least six months when stored in the dark at room temperature. The synergistic composition may contain water soluble B vitamins and vitamin C, fat soluble vitamins A, D, E, K and essential trace elements. The composition additionally may contain one or more of chondroitin sulphate, glucosamine sulphate, glutamine, choline, arginine, trace elements like Zinc, Iron (Fe) and Se, N acetyl cysteine, leutine, lycopene, alpha lipoic acid Q-10, curcumin and, quercitine.

The following experimental procedures were employed in the invention:
1. The stability of the oil was determined by TBAR assay of lipid peroxide and expressed as n moles of MDA (malanodialdehydes) (Ohkawa et al et al Anal Biochem, 95, 1979, 351).
2. Omega-3 fatty acids estimated by GC (Meena Arvindakshan et al, Schiz Res, 62, 2003, 195).
3. Vitamin E—estimated spectrophotometrically (Barker and Frank et al Clinical Vitaminolgy 1968, 172).
4. Vitamin C estimated spectrophotometrically (Ayekyaw, Clinica Chmica Acta, 86, 1978, 153).
5. Attributes of Honey were analyzed and certified by Central Bee Research Institute, Pune.

Very high ALA containing flax seed varieties were selected, cleaned, and polished. Oil extraction was carried out in cold press, filtered and stored in a cold room, without any air contact, until use. Oil is used within a week. Flax oil had 55-60% ALA. For preparing a composition, of the invention, natural or organic honey is used. Organic Honey has not only, antibacterial and antifungal activity, but also in addition, honey naturally contains several antioxidants. Many antioxidants identified in honey include phenolics, ascorbic acid, hydroxymethyl fufuraldehyde and the combined activities of enzymes glucose oxidase, catalase and peroxidases. Table 2 below provides the attributes of the different honey used in the nutraceutical composition in this invention.

TABLE 2
attributes of different honey used in nutraceutical compositions

| Attributes | I | II | III | IV | V |
|---|---|---|---|---|---|
| Variety | Jambul | Gela | Karvi | Litchi | Melghat |
| Location | Mahabaleshwar | Mahabaleshwar | Mahabaleshwar | Muzaffarpur | Melghat |
| Uni/Multifloral | unifloral | unifloral | unifloral | unifloral | Multifloral |
| | | | | | Butea frondosa, |
| | | | | | Butea monosperma, |
| Foraging source | Syzygium | Xeromphis | Carvia | Litchi | Madhuca sp., |
| | jambolana | spinosa | callosa | chinensis | Aconitum sp |
| | | | | | Leguminaceae, |
| Family | Myrtaceae | Rubiaceae | Acanthaceae | Sapindaceae | Sapotaceae |
| Age when | 3 months | 3 months | 1 year | 4 months | 4 months |
| Collected | | | | | |
| Availability | annual | annual | once in eight yrs | annual | throughout the |
| | | | | | yr (except monsoon) |
| Bee species | Apis cerana | Apis cerana | Apis cerana | Apis cerana | Apis dorsata |
| Processing | Traditional | Traditional | Unprocessed | Modern | Unprocessed |
| Method | | | | (CBRTI) | |
| Colour | Dark amber | Amber | Dark amber | Light amber | Dark amber |
| Aroma | Floral | Floral | Floral | Fruity | Floral, Heady |
| Flavor | Sweet | Sweet | Sweet | Fruity | Sweet, intoxicating |
| Specific gravity | 1.37 | 1.408 | 1.4 | 1.388 | 1.46 |
| Moisture % | 17.6 | 21.8 | 27 | 23.5 | 20 |
| Total reducing | 76.76 | 75.05 | 75.91 | 74.43 | 67.12 |
| sugars | | | | | |
| F/G ratio | 1.15 | 0.95 | 1.08 | 1.06 | 1.35 |
| Sucrose % | 2.86 | 0.99 | 5.08 | 0.6 | 2.03 |
| Acidity (Formic acid | 0.08 | 0.08 | 0.15 | 0.07 | 0.15 |
| %) | | | | | |
| Fiehes test | Negative | Negative | Negative | Negative | Negative |
| Aniline chloride | Negative | Negative | Negative | Negative | Negative |
| Lipid peroxides | 5.03 | 10.26 | — | 3.97 | 5.57 |
| (MDA nMol) | | | | | |
| Hydroxyl-Methyl | 11.06 | 19.04 | 2.30 | 3.53 | 16.28 |
| furfaral | | | | | |
| Vitamin C mg % | 1.92 | 1.97 | 2.5 | 0.6 | 1.96 |

The honey selected is locally sourced. The varieties called Jamun, Gela, Karvi, and Litchi, are unifloral honey, whereas Melghat is multifloral honey. They are sourced from different locations in India as indicated in the table. They all have different foraging source and distinctive flavour and compositions. Bee species for unifloral is Apis cerana and for multifloral honey it is Apis dorsata. They are either traditional or unprocessed. They possess sweet, fruity to intoxicating flavors. They have specific gravity 1.37 to 1.46 with moisture content of 17-27%. Although the preparation is described with reference to locally sourced honey it is only for the purpose of description of the invention and the invention is not limited to selection of a local variety of honey.

For the preparation of omega-3 honey formulations, honey with a lower moisture content is preferred. Total reducing sugar is 67-77%, with fructose to glucose ratio ranging form 0.95 to 1.35.

Hydroxy methyl furfural content of different honey ranges from 2.3 to 19.0. The lipid peroxides in honey is less than 10.26 n moles MDA per ml of honey. Vitamin C content of different honey ranges from 0.6 to 2.5 mg %.

All of them were analyzed and are certified by Central Bee Research and Training Institute, Pune, an Institute of the Government.

The natural antioxidants used such as Vitamin E, Vitamin C etc. may be sourced from commercial preparations.

In one method of preparation, a first pre-blend of all liquid ingredients inclusive of the oil is prepared. A second blend of dry ingredients is also prepared, a third blend of honey and one of the dry ingredients is separately prepared. One of the liquids is initially stabilized at refrigerated temperature of 6° C. The dry ingredient blend is mixed with the honey blend. Thereafter the liquid blend is added and the product is conventionally homogenized to a droplet size of approx. 10 micron size for phase stability.

In another preferred method of preparation, the formulation is as described above except that all the dry ingredients are premixed with honey and then treated with the liquid ingredient inclusive of oil and then mixed. Further the homogenization is performed at a lower speed so that the droplet size is around 10-20 microns.

Use of antioxidants imparts protection against an increase in lipid peroxide levels. It is necessary to add natural antioxidants in the composition for assimilation of the composition in the body. The natural antioxidants used may be Vitamin E, Vitamin C etc. As the composition contains both, a fatty acid component and a water component, both oil soluble and water soluble vitamins can be added.

Without further elaboration, it is believed that one skilled in the art can use the preceding description to, utilize the present invention to its fullest extent. The following preferred specific embodiments are therefore to be construed as merely illustrative and not limitative of the remainder of the disclosure whatsoever.

For the examples given below the following procedure was generally used with suitable variations.

About 200 gm of formulation was prepared making a pre-blend of all dry ingredients and a second pre-blend of liquid ingredients. There after the dry pre-blend was mixed with one of the liquid pre-blend using a mortar and pestle thereafter the composition was blended at slightly higher speed manually and the other liquid ingredient mixed thoroughly. The whole mixture was then blended in a wet grinder to form a stable emulsion.

In the formulations described in various examples below, flax oil is emulsified with different varieties of honey mentioned above, using surfactants such as Polysorbate (40, 60, 80), Pluronic acid etc. and natural antioxidants.

Formulations of comparable attributes and omega-3 oil stability are obtained when the Gela honey is replaced with uni-floral or multi-floral honey. Different flavors like chocolate and cardamom when incorporated produced similar results.

Formulations of comparable organoleptic attributes and omega-3 oil stability are obtained when the flavors are substituted with fresh fruit powders like pineapple, mango, and banana, others like chocolate, butterscotch. Results of shelf life studies of orange saffron and pineapple-flavored formulations are presented here.

Some examples of different compositions are given below:

### EXAMPLE 1

### OS

| Ingredients | Weight % |
|---|---|
| Omega-3 Oil | 20.24 |
| Honey (Gella) | 76.8 |
| Emulsifier (Polysorbate 40, 60, 80) | 2.1 |
| Antioxidant blend | 0.3 |
| Sodium Methyl Cellulose | 0.19 |
| Orange Saffron Flavor | 0.37 |
| Total | 100 |

### EXAMPLE 2

### PN

| Ingredients | Weight % |
|---|---|
| Omega-3 Oil | 20.24 |
| Honey (Litchi) | 76.8 |
| Emulsifier (Polysorbate 40, 60, 80) | 2.20 |
| Antioxidant blend | 0.25 |
| Sodium Methyl Cellulose | 0.18 |
| Pineapple Flavor | 0.33 |
| Total | 100 |

### EXAMPLE 3

### GT-9

| Ingredients | Weight % |
|---|---|
| Omega-3 Oil | 19.32 |
| Jamun Honey | 74.4 |
| Emulsifier | 6.00 |
| (Polysorbate 40, 60, 80) | |
| Antioxidant blend | 0.22 |
| Pineapple Flavor | 0.06 |
| Total | 100 |

### EXAMPLE 4

### GT-8

| Ingredients | Weight % |
|---|---|
| Omega-3 Oil | 19.32 |
| Honey (Multifloral) | 74.4 |
| Emulsifier (Polysorbate 40, 60, 80) | 6.00 |
| Antioxidant blend | 0.22 |
| Pineapple Flavor | 0.06 |
| Total | 100 |

### EXAMPLE 5

### GEL-1

| Ingredients | Weight % |
|---|---|
| Omega-oil | 27.60 |
| Honey (Jamun) | 66.00 |
| Emulsifier 1 (Polysorbate 40, 60, 80) | 2.30 |
| Emulsifier 2 Pluronic | 4.00 |
| Antioxidant blend | 0.10 |
| Total | 100.00 |

### EXAMPLE 6

### GEL-7

| Ingredients | Weight % |
|---|---|
| Omega-3 Oil | 24.84 |
| Honey | 66.00 |
| Emulsifier 1 | 1.16 |
| (Milk powder) | |
| Emulsifier 2. | 5.40 |
| Pluronic acid | |
| Antioxidant blend | 0.36 |
| Pineapple powder | 2.24 |
| Total | 100 |

### EXAMPLE 7

### GT-6

| Ingredients | Weight % |
|---|---|
| Omega-3 Oil | 27.60 |
| Honey (Multifloral) | 66.00 |
| Emulsifier 1 | 2.00 |
| (Polysorbate 40, 60, 80) | |
| Antioxidant blend | 0.26 |
| Emulsifier 2-Pluronic acid | 4.14 |
| Total | 100 |

### EXAMPLE 8

### Gel-10

| Ingredients | Weight % |
|---|---|
| Omega-3 Oil | 24.84 |
| Honey (Multifloral) | 66.00 |
| Emulsifier 1. | 2.00 |
| (milk powder + Polysorbate 60, 80) | |
| Antioxidant blend | 0.16 |
| Emulsifier 2: Pluronic acid | 7.00 |
| Total | 100 |

### EXAMPLE 9

### MP-1

| Ingredients | Weight % |
|---|---|
| Omega-3 Oil | 25.76 |
| Honey (Multifloral) | 66.00 |
| Emulsifier 1. (Polysorbate 40, 60, 80) | 2.00 |
| Antioxidant blend | 0.22 |
| Emulsifier 2-Pluronic acid | 6.02 |
| Total | 100 |

### EXAMPLE 10

### IQ

| Ingredients | Weight % |
|---|---|
| Omega-3 Oil | 10.12 |
| Honey (Multifloral) | 84.00 |
| Emulsifiers | 3.00 |
| (Maltodextrin and a surfactant) | |
| Antioxidant blend | 0.16 |
| Pineapple Fruit Powder (Spray dried) | 2.25 |
| Pineapple Flavor | 0.47 |
| Total | 100 |

### EXAMPLE 11

### SE 1 RT (SE 1 CT (Cold Temp at 6° C.)

| Ingredients | Weight % |
|---|---|
| Omega-3 Oil | 30.36 |
| Honey (Multifloral) | 66.00 |
| Emulsifiers | 3.00 |
| (Maltodextrin/and a surfactant) | |
| Antioxidant blend | 0.61 |
| Pineapple Flavor | 0.03 |
| Total | 100 |

The composition of a typical preparation is presented in Table 3

TABLE 3
Composition of a typical preparation (Quantity - 10 GMS)

| TOTAL CARBOHYDRATES | 7.75 g |
|---|---|
| TOTAL FAT | 1.84 g |
| OMEGA-3 FATTY ACIDS | 0.80 g |
| TOTAL ANTIOXIDANTS (E + C) | 0.45 g |
| TOTAL CALORIES | 46.56 |

In another embodiment, the composition can also be used as vehicle to incorporate certain specific herbs that have known medicinal properties. The use of omega-3 formulations for treatment of various diseases is well documented in prior art. The method can also be used to incorporate all the essential nutrients: water and fat soluble vitamins, essential amino acids, essential minerals and the essential fatty acids etc. to make it a complete nutritional supplement.

In all above compositions described in the Examples, there was very little peroxidation, indicating that in the formulations with honey, omega-3 is very well stabilized. The power of this stabilization in this formulation surpasses the effect of natural vitamin by several folds. The various constituents of the composition act synergistically. The compositions are stable at least for a period of six months.

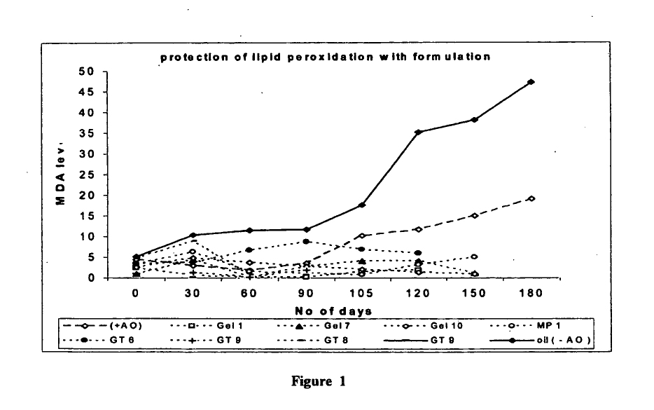

This is reflected in FIG. 1. The FIG. 1 gives the lipid peroxidation values (LPO) of some of the compositions from the examples over a period of 5-6 months. Oil (−AO) represents flax oil to which no additional antioxidants have been added and it deteriorates very fast and has the highest LPO values. Addition of vitamin E (+AO) does provide some protection, but it also starts deteriorating after about 60 days. But the formulations with honey are stable throughout.

While particular embodiments of the invention are described here, the present invention however is not limited to any particular application or environment. Modifications and variations may occur to those skilled in the art. The description of the exemplary embodiments, is therefore, for the purpose of illustration and not limitation.

### INDUSTRIAL APPLICABILITY

The compositions disclosed in the invention are useful to supply needs of Omega-3 fatty acids. The composition may be used directly or by enrichment with other ingredients as disclosed. The compositions can be used as base for preparing herbal compositions.

1-15. (canceled)

16. A process for preparing a synergistic and stable composition of omega-3 fatty acids, honey and antioxidants comprising the steps of:
a) extracting omega-3 fatty acids from a source containing omega-3-fatty acids;
b) emulsifying the omega-3 fatty acids with honey using one or more surfactants; and
c) enriching the omega-3 fatty acids and honey mixture with antioxidants to obtain the composition.

17. The process according to claim 16, further comprising adding a flavor to the composition.

18. The process as claimed in claim 16, wherein the omega-3 fatty acids are extracted from a plant, algae, fish or an egg or any biological source or any combination thereof.

19. The process as claimed in claim 16, wherein the omega-3 fatty acids are extracted from flax seeds.

20. The process as claimed in claim 1, wherein the honey has a moisture content of 17-27%.

21. The process as claimed in claim 20, wherein honey comprises reducing sugar and the reducing sugar in the honey is between 67-77%.

22. The process as claimed in claim 20, wherein the honey comprises fructose and glucose and the fructose to glucose ratio is between 0.95-1.35.

23. The process as claimed in claim 16, wherein the honey is unifloral or multifloral.

24. The process as claimed in claim 16, wherein the surfactant is Polysorbate (40, 60, 80), Pluronic acid, or a mixture thereof.

25. The process as claimed in claim 16, wherein the honey contains natural antioxidants.

26. The process as claimed in claim 24, wherein the antioxidants are selected from Vitamin E and Vitamin C or a mixture thereof.

27. The process as claimed in claim 16, wherein the antioxidant comprises water or lipid soluble antioxidants.

28. The process as claimed in claim 17, wherein the flavor is selected from the group consisting of chocolate, fruit flavors, cardamon, and butterscotch or a combination thereof.

29. A synergistic and stable composition of omega-3 fatty acids with honey and natural antioxidants containing stabilized omega-3 fatty acids.

30. The synergistic composition as claimed in claim 29, being in a stabilized and emulsified state.

31. The synergistic composition as claimed in claim 30, being in a stabilized and emulsified state for at least six months.

32. The synergistic composition as claimed in claim 30, further comprising one or more of water soluble B vitamins, vitamin C, fat soluble vitamins A, D, E, or K or essential trace elements.

33. The composition as claimed in claim 30, further comprising one or more of chondroitin sulphate, glucosamine sulphate, glutamine, choline, arginine, Zinc, Iron (Fe), Se, N acetyl cysteine, leutine, lycopene, alpha lipoic acid Q-10, curcumin, and quercitine.

34. The composition according to claim 29, further comprising a flavoring agent selected from the group consisting of chocolate, fruit flavors, cardamon and butterscotch or a combination thereof.

35. The composition according to claim 34 that is a beverage.
